SEQUENCE LISTING (6 sequences)

SEQ ID NO: 1 - DNA, 800 bp, Artificial Sequence
  homology arm
ttttgctgccagttgacatctgattgaaccatctcttcacttctccgtgcctcactttccttaccagacaggctctgctgatgctgtccctctcctgttcagtcgtgccctcaccgttaaagagaaagagcaaactgctgggcagcagcattgatttttttaatgaagtggaaagagagctgggaataacaagtcgggcccacctcacctgcctcacctggtgggtttatttgttttgtttttttttttttgttttgagacagagtttcaccctgtcacccaggctggagtgcagtggtgtaatctcagctcactgcaacctccacctgccaggttcaattgattctcctgcctcagcctccccagtagctgggattacaggcacctgccacatgcctggctaattattgtatttttagtagagatggggttttaccatgttggccaggctggtctcgatcccctgacctcaggtgatccacccacctcggcctcccaaagtgctgagatcacaggcgtgagccaccatgcctggccgtcacctggtggtgttgaatatgaactgctgcggtgttggtaaattaagcaagcagatagatgtaaataacgcttgggcaggaatatggagcacgggatgaggatgggcggccaactgttagagagggtagcagggaggctgagatctgcctgccatgaactgggaggagaggctcctctctctcttcacccccactctgccccccaacactcctcagaacttatcctctcctcttctttccccaggtgaactttgaaccagg

SEQ ID NO: 2 - DNA, 800 bp, Artificial Sequence
  homology arm
atggctgagccccgccaggagttcgaagtgatggaagatcacgctgggacgtacgggttgggggacaggaaagatcaggggggctacaccatgcaccaagaccaagagggtgacacggacgctggcctgaaaggttagtggacagccatgcacagcaggcccagatcactgcaagccaaggggtggcgggaacagtttgcatccagaattgcaaagaaattttaaatacattattgtcttagactgtcagtaaagtaaagcctcattaatttgagtgggccaagataactcaagcagtgagataatggccagacacggtggctcacgcctgtaatcccagcactttggaaggcccaggcaggaggatcccttgaggccaggaatttgagaccggcctgggcaacatagcaagaccccgtctctaaaataatttaaaaattagccaggtgttgtggtgcatgtctatagtcctagctactcaggatgctgaggcagaaggatcacttgagcccaggagttcaaggttgcagtaagctgtgattataaaactgcactccagcctgagcaacagagcaagaccctgtcaaaaaaaaaagaaaagaaaaaagaaagaaagaaatttaccttgagttacccacatgagtgaatgtagggacagagattttagggccttaacaatctctcaaatacagggtactttttgaggcattagccacacctgttagcttataaatcagtggtattgattagcatgtaaaatatgtgactttaaacattgctttttatctcttacttagatc

SEQ ID NO: 3 - DNA, 800 bp, Artificial Sequence
  homology arm
atactaagcagcctgtgtatctatacactcacacgtgtttgtttatgtgtggaatatctctggagggtacacaagaaacttaaaatgatcactgtctctggggagggtacctgggtgcctgggaggcaggtcagggaaggagtgggcacaggtattaccaattggaagacaataaaaacaacagctcctggccaggcgcagtggctcacgcctgtaatggcagcactctgagaggctgaggcgggcagattgcttgcgtccaggagttcaagaccagcctgggcaacatagcaaaaccccgtttctattaaaaatacaaaaaattagccaggtgtggtggcatgcacctgtaatcccagctactcgggaggctgaggtgggagaatcacctgagcctgggaggtcaaggctgcagtgaggtgagattgtgccaccgcactctagcctgggcgatagagcaagaccctgtctcaaaaacaaacaaaaaacagtccctggcactctgggccaggcctggcagggcagttggcagggctggtctttctctggcacttcatctcaccctccctcccttcctcttcttgcagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttg

SEQ ID NO: 4 - DNA, 800 bp, Artificial Sequence
  homology arm
tcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtg

SEQ ID NO: 5 - DNA, 20 bp, Artificial Sequence
  target sequence of sgRNA
aggtgaactttgaaccagga

SEQ ID NO: 6 - DNA, 20 bp, Artificial Sequence
  target sequence of sgRNA
acaattattgaccgccccag

(19) United States
(12) Patent Application Publication
(10) Pub. No.: US 2022/0326225 A1
(43) Pub. Date: Oct. 13, 2022

(54) PLURIPOTENT STEM CELL, NERVE CELL, AND APPLICATION THEREOF

(71) Applicants: FUJIFILM Corporation, Tokyo (JP); KEIO UNIVERSITY, Tokyo (JP)

(72) Inventors: Hirokazu TANABE, Ashigarakami-gun (JP); Setsu ENDOH, Ashigarakami-gun (JP); Hitoshi MASUYAMA, Ashigarakami-gun (JP); Euikyung SHIN, Ashigarakami-gun (JP); Koichi SAITO, Ashigarakami-gun (JP); Hideyuki OKANO, Tokyo (JP); Sumihiro MAEDA, Tokyo (JP)

(73) Assignees: FUJIFILM Corporation, Tokyo (JP); KEIO UNIVERSITY, Tokyo (JP)

(21) Appl. No.: 17/709,903

(22) Filed: Mar. 31, 2022

(30) Foreign Application Priority Data
Mar. 31, 2021 (JP) 2021-061793

(51) Int. Cl. G01N 33/50; C12Q 1/6897; C12N 5/0793; C12N 15/62; C12N 15/11; C12N 9/22; C12N 15/86; C07K 14/47

(52) U.S. Cl. CPC G01N 33/5058; C12Q 1/6897; C12N 5/0619; C12N 15/62; C12N 15/11; C12N 9/22; C12N 15/86; C07K 14/47; C12N 2506/45; C12N 2501/13; C12N 2310/20; C12N 2800/80; C12N 2740/15022; C12N 2740/15042; C12N 2740/15062; C07K 2319/00

(57) ABSTRACT

Objects to be achieved are to provide a nerve cell with which it is possible to visualize and quantify the intracellular tau without using the exogenous promoter and to provide a pluripotent stem cell with which the nerve cell can be produced, to provide a method of screening a substance, including using the pluripotent stem cell or nerve cell described above, and a substance screened by the above method, and to provide a kit including a targeting vector and a gRNA.

There is provided a pluripotent stem cell including a DNA encoding a reporter molecule, the DNA being introduced adjacent to an endogenous tau gene such that a tau protein is expressed as a fusion protein fused with a reporter molecule.

### CROSS-REFERENCE TO RELATED APPLICATIONS

This application claims priority under 35 U.S.C 119 to Japanese Patent Application No. 2021-061793 filed on Mar. 31, 2021. Each of the above applications is hereby expressly incorporated by reference, in its entirety, into the present application.

### BACKGROUND OF THE INVENTION

### 1. Field of the Invention

The present invention relates to a pluripotent stem cell that is modified so that a tau protein is expressed as a fusion protein fused with a reporter molecule. In addition, the present invention relates to a nerve cell differentiated from the pluripotent stem cell. Further, the present invention relates to a method of screening a substance, including using the pluripotent stem cell or nerve cell described above, and a substance screened by the above method. Further, the present invention relates to a kit including a targeting vector and a gRNA.

### 2. Description of the Related Art

Since the development mechanism of a neurodegenerative disease such as Alzheimer's disease is diverse, the research and development of therapeutic drugs based on various mechanisms are expected. Tau is conceived to be a major factor involved in the development of the neurodegenerative disease and increases in the brain of a patient with a neurodegenerative disease. In addition, it has been reported that there is a correlation between the amount of tau accumulated and the decrease in cognitive function, and thus tau is expected as a therapeutic target for neurodegenerative diseases. However, it is still clear how tau accumulation is caused, and how the tau accumulation should be controlled to lead to the treatment thereof, what happens after the tau accumulation, and how the tau accumulation should be controlled. Based on these backgrounds, as a research tool for drug discovery targeting tau, it has been desired to establish human nerve cells with which it is possible to visualize and quantify the intracellular increase or decrease and localization change of tau.

There is a report on a cell evaluation system with which the intracellular tau is visualized and quantified using a fusion gene of a tau fluorescent protein. For example, Human Molecular Genetics, 2015, Vol. 24, No. 14 3971-3981, discloses a method of quantifying the co-localizability of tau and microtubules by RFP fluorescence and a method of quantifying an RFP-Tau protein by western blotting, in which an RFP-Tau gene is introduced from the outside into a non-nerve cell (HEK293T) that does not express tau, RFP-Tau is artificially expressed under exogenous promoter control. In Human Molecular Genetics, 2015, Vol. 24, No. 14 3971-3981, in addition to the cell being a non-nerve cell, the protein expression is an artificial expression by an exogenous promoter, and thus the expression in the nerve cell depending on the promoter of the tau gene itself or the expression or distribution of tau in the nerve cell is not measured. J Neurosci. 2017 Nov. 22; 37 (47): 11485-11494 discloses that a mutant Tau-GFP is introduced into a mouse, the expression thereof is promoted by an exogenous promoter, and the localization and the expression level of tau in the mouse brain are visualized by GFP fluorescence. Neuron. 2007 Feb. 1; 53 (3): 337-51 also discloses that tau is forcibly expressed by an exogenous promoter.

Stem Cell Reports. 2017 Oct. 10; 9 (4): 1221-1233 discloses that a nerve cell is prepared from a human iPS cell by expressing Ngn2 (through a Tet-on system). In Stem Cell Reports. 2017 Oct. 10; 9 (4): 1221-1233, the expressed tau is quantified by fluorescent staining.

WO2008/102903A discloses a system for evaluating the presence or absence of splicing at a specific site of a specific gene in a cell line based on the expression of GFP.

### SUMMARY OF THE INVENTION

Regarding tau which is expressed using an exogenous promoter as described in Human Molecular Genetics, 2015, Vol. 24, No. 14 3971-3981. J Neurosci. 2017 Nov. 22; 37 (47): 11485-11494, and Stem Cell Reports. 2017 Oct. 10; 9 (4): 1221-1233, the distribution or function thereof in the cell may be different from that in the human body. In addition, since the structure of the tau protein and the expressed isoform thereof are different between mice and humans, the mode of binding and release of the tau inside and outside the nerve cell may be also different. Furthermore, in a model mouse with Alzheimer's disease, it has been reported that human nerve cells are more vulnerable to neurotoxic substances than murine nerve cells and that tau pathology is strongly exhibited, and thus the response of nerve cells through tau may differ depending on the organism. For these reasons, it has been expected to establish a cell with which it is possible to visualize the tau expressed under the control of the endogenous tau and simply evaluate the expression level and the localization of the tau in a human nerve cell. In addition, fluorescent staining is used in Stem Cell Reports. 2017 Oct. 10; 9 (4): 1221-1233, and thus there is a high possibility that it is difficult to control the variation in the staining operation (cell detachment or the like) and all tau molecular species are not visualized due to the specificity of the antibody. Furthermore, the accuracy of quantification depends on the properties of the antibody.

An object to be achieved in the present invention is to provide a nerve cell with which it is possible to visualize and quantify the intracellular tau without using the exogenous promoter and to provide a pluripotent stem cell with which the nerve cell can be produced. In addition, another object to be achieved in the present invention is to provide a method of screening a substance, including using the pluripotent stem cell or nerve cell described above, and a substance screened by the above method. Further, another object to be achieved in the present invention is to provide a kit including a targeting vector and a gRNA.

As a result of diligent studies to achieve the above objects, the inventors of the present invention first introduced a DNA encoding a reporter molecule, adjacent to an endogenous tau gene, to prepare a pluripotent stem cell in which a tau protein could be expressed as a fusion protein fused with a reporter molecule. Next, the inventors of the present invention succeeded in producing a nerve cell with which it was possible to visualize and analyze the intracellular tau, by differentiating the pluripotent stem cell into a nerve cell. The present invention has been completed based on the above findings.

That is, according to the present invention, the following inventions are provided.

<1> A pluripotent stem cell comprising a DNA encoding a reporter molecule, the DNA being introduced adjacent to an endogenous tau gene such that a tau protein is expressed as a fusion protein fused with a reporter molecule.

<2> The pluripotent stem cell according to <1>, in which the pluripotent stem cell is a human pluripotent stem cell.

<3> The pluripotent stem cell according to <1> or <2>, in which the pluripotent stem cell is an induced pluripotent stem cell.

<4> The pluripotent stem cell according to any one of <1> to <3>, in which the reporter molecule is a fluorescent protein.

<5> The pluripotent stem cell according to any one of <1> to <4>, in which the DNA encoding the reporter molecule is located upstream of the endogenous tau gene.

<6> A nerve cell differentiated from the pluripotent stem cell according to any one of <1> to <5>.

<7> The nerve cell according to <6>, in which a fusion protein of a tau protein and a reporter molecule is expressed.

<8> A nerve cell comprising a DNA encoding a reporter molecule, the DNA being introduced adjacent to an endogenous tau gene such that a tau protein is expressed as a fusion protein fused with a reporter molecule.

<9> The nerve cell according to <8>, in which the nerve cell is an established nerve cell line or a primary nerve cell.

<10> The nerve cell according to <8> or <9>, in which the reporter molecule is a fluorescent protein.

<11> The nerve cell according to any one of <8> to <10>, in which the DNA encoding the reporter molecule is located upstream of the endogenous tau gene.

<12> A method of screening a substance, comprising using the pluripotent stem cell according to any one of <1> to <5> or the nerve cell according to any one of <6> to <11>.

<13> The method according to <12>,

in which an evaluation of an increase or decrease in an expression level of tau or an evaluation of an intracellular distribution of tau is carried out based on an expression of a reporter molecule.

<14> The method according to <12> or <13>, in which an increase or decrease in an expression level of tau is evaluated based on an expression intensity of a reporter molecule.

<15> A substance screened by the method according to any one of <12> to <14>.

<16> A kit comprising:

a targeting vector that includes homology arms upstream and downstream of a tau gene insertion site and includes a DNA encoding a reporter molecule; and

a gRNA that determines a cleavage site of the tau gene.

<17> The kit according to <16>,

in which the homology arm upstream of the tau gene insertion site is a sequence having 90% or more identity with a sequence set forth in SEQ ID NO: 1, and the homology arm downstream of the tau gene insertion site is a sequence having 90% or more identity with a sequence set forth in SEQ ID NO: 2, or

the homology arm upstream of the tau gene insertion site is a sequence having 90% or more identity with a sequence set forth in SEQ ID NO: 3, and the homology arm downstream of the tau gene insertion site is a sequence having 90% or more identity with a sequence set forth in SEQ ID NO: 4.

<18> The kit according to <17>, in which the gRNA targets a sequence having 90% or more identity with a sequence set forth in SEQ ID NO: 5 or 6.

According to the pluripotent stem cell and the nerve cell according to aspects of the present invention, it is possible to evaluate the intracellular expression level and distribution of the endogenous tau. According to the pluripotent stem cell and the nerve cell according to aspects of the present invention, the introduction of the exogenous tau gene is not required, and it is possible to evaluate the expression of the endogenous tau without using an exogenous promoter.

### BRIEF DESCRIPTION OF THE DRAWINGS

FIG. 1 is a diagram illustrating a TagGFP2 fusion to the N-terminus of an MAPT gene by CRISPR-Cas9.

FIG. 2 a diagram illustrating a TagGFP2 fusion to the C-terminus of an MAPT gene by CRISPR-Cas9.

FIG. 3 is a map showing a CSIV-miR-9/9*-124-mRFP1-TRE-EF-BsdT vector.

FIG. 4 is a schematic diagram illustrating a vector used in the nerve cell induction.

FIG. 5 is images showing TagGFP2-Tau fluorescence in iPS cell-derived nerve cells.

FIG. 6 is graphs showing results of analyzing fluorescent Tau quantification and neurite length in bright field.

### DESCRIPTION OF THE PREFERRED EMBODIMENTS

Hereinafter, the content of the present invention will be described in detail.

In the present specification, “NP_”, “NM_”, and “NG_” together with numbers following them respectively represent an amino acid sequence (NP_˜), a nucleotide sequence (NM_˜) of a transcript, and an ID of a genomic DNA sequence (NG_˜), which are registered as the reference sequences in the National Center for Biotechnology Information (NCBI) database.

Pluripotent Stem Cell

The pluripotent stem cell according to the embodiment of the present invention has a DNA encoding a reporter molecule, the DNA being introduced adjacent to an endogenous tau gene such that a tau protein is expressed as a fusion protein fused with a reporter molecule.

The “pluripotent stem cell” refers to a cell having the ability (the differentiation pluripotency) to differentiate into all cells that constitute a living body and the ability (the self-replication ability) to generate a daughter cell having the same differentiation potency as the mother cell through cell division. The differentiation pluripotency can be evaluated by transplanting an evaluation target cell into a nude mouse and testing for the presence or absence of formation of teratoma that includes cells of the respective three germ layers (ectoderm, mesoderm, and endoderm).

Examples of the pluripotent stem cell include an embryonic stem cell (an ES cell), an embryonic germ cell (an EG cell), and an induced pluripotent stem cell (an iPS cell); however, examples thereof are not limited thereto as long as a cell has both differentiation pluripotency and self-replication ability. An ES cell or an iPS cell is preferably used. An iPS cell is more preferably used. The pluripotent stem cell is preferably a mammalian (for example, primates such as a human or a chimpanzee, rodents such as a mouse or a rat) cell. The pluripotent stem cell is particularly preferably a human pluripotent stem cell. In the most preferred aspect of the present invention, a human iPS cell is used as the pluripotent stem cell.

The ES cell can be established, for example, by culturing an early embryo before implantation, an inner cell mass constituting the above early embryo, or a single blastomere (Manipulating the Mouse Embryo, A Laboratory Manual, Second Edition, Cold Spring Harbor Laboratory Press (1994); Thomason, J. A. et al., Science, 282, 1145-1147 (1998)). As the early embryo, an early embryo prepared by nuclear transfer of a somatic cell nucleus may be used (Wilmut et al. (Nature, 385, 810 (1997)), Cibelli et al. (Science, 280, 1256) (1998)), Akira Iriya et al. (Protein, nucleic acid, and enzyme, 44, 892 (1999)), Baguisi et al. (Nature Biotechnology, 17, 456 (1999)), Wakayama et al. (Nature, 394, 369 (1998)); Nature Genetics, 22, 127 (1999); Poc. Natd. Acad. Sci. USA, 96, 14984 (1999)), Rideout III et al. (Nature Genetics, 24, 109 (2000), Tachibana et al. (Human Embryonic Stem Cells Delivered by Somatic Cell Nuclear Transfer, Cell (2013) in press). As the early embryo, a parthenogenetic embryo may be used (Kim et al. (Science, 315, 482-486 (2007)), Nakajima et al. (Stem Cells, 25, 983-985 (2007)), Kim. et al. (Cell Stem Cell, 1,346-352 (2007)), Revazova et al. (Cloning Stem Cells, 9, 432-449 (2007)), Revazova et al. (Cloning Stem Cells, 10, 11-24 (2008)). In addition to the above-described papers, regarding the preparation of an ES cell, the following can be referenced, Strelchenko N. et al. Reprod Biomed Online. 9: 623-629, 2004; Klimanskaya I., et al. Nature 444: 481-485, 2006; Chung Y., et al. Cell Stem Cell 2: 113-117,2008; Zhang X., et al. Stem Cells 24: 2669-2676, 2006; Wassarman, P. M. et al. Methods in Energy, Vol. 365, 2003, and the like. It is noted that a fused ES cell obtained by cell fusion of an ES cell with a somatic cell is also included in the embryonic stem cell that is used in the method according to the embodiment of the present invention.

Some ES cells are available from conservation institutions or are commercially available. For example, human ES cells are available from the Institute for Frontier Medical Sciences, Kyoto University (for example, KhES-1, KhES-2, and KhES-3), WiCell Research Institute, ESI BIO, and the like.

The EG cell can be established by, for example, culturing a primordial germ cell in the presence of a leukemia inhibitory factor (LIF), a basic fibroblast growth factor (bFGF), and a stem cell factor (SCF) (Matsui et al., Cell, 70, 841-847 (1992), Shamblott et al., Proc. Natl. Acad. Sci. USA, 95 (23), 13726-13731 (1998), Turnpenny et al., Stem Cells, 21 (5), 598-609, (2003)).

“Induced pluripotent stem cell (iPS cell)” is a cell having pluripotency (multiple differentiation potency) and proliferation ability, which is prepared by reprogramming a somatic cell by introducing reprogramming factors or the like. The induced pluripotent stem cell exhibits properties similar to the ES cell. The somatic cell that is used for preparing an iPS cell is not particularly limited and may be a differentiated somatic cell or an undifferentiated stem cell. In addition, the origin of the somatic cell is not particularly limited: however, a somatic cell of a mammal (for example, primates such as a human or a chimpanzee, rodents such as a mouse or a rat) cell is preferably used, and a human cell particularly preferably used. The iPS cell can be prepared by various methods reported so far. In addition, it is naturally expected that an iPS cell preparation method to be developed in the future will be applied.

The most basic method of preparing an iPS cell is a method of introducing four transcription factors, Oct3/4, Sox2, Klf4, and c-Myc, into a cell using a virus (Takahashi K, Yamanaka S: Cell 126 (4), 663-676, 2006; Takahashi, K, et al: Cell 131 (5), 861-72, 2007). It has been reported that human iPS cells have been established by introducing four factors, Oct4, Sox2, Lin28, and Nanog (Yu J, et al.: Science 318 (5858), 1917-1920, 2007). It has also been reported that iPS cells are established by introducing three factors excluding c-Myc (Nakagawa M, et al: Nat. Biotechnol. 26 (1), 101-106, 2008), two factors of Oct3/4 and Klf4 (Kim J B, et al: Nature 454 (7204), 646-650, 2008), or only Oct3/4 (Kim J B, et al: Cell 136 (3), 411-419, 2009). In addition, a method of introducing a protein, which is an expression product of a gene, into a cell (Zhou H, Wu S, Joo J Y, et al: Cell Stem Cell 4, 381-384,2009; Kim D, Kim C H, Moon J I, et al.: Cell Stem Cell 4, 472-476, 2009) has also been reported. On the other hand, it has been also reported that it is possible to improve the preparation efficiency or reduce the factors to be introduced, by using an inhibitor BIX-01294 for a histone methyltransferase G9a, a histone deacetylase inhibitor valproic acid (VPA), Bay K8644, or the like (Huangfu D, et al: Nat. Biotechnol. 26 (7), 795-797, 2008; Huangfu D, et al: Nat. Biotechnol. 26 (11), 1269-1275, 2008; Silva J, et al: PLoS. Biol. 6 (10), e253, 2008). In addition, gene introducing methods have been studied as well, and techniques using, in addition to retroviruses, the following substances have been developed; lentiviruses (Yu J, et al: Science 318 (5858), 1917-1920, 2007), adenoviruses (Stadtfeld M, et al: Science 322 (5903), 945-949, 2008), plasmids (Okita K, et al: Science 322 (5903), 949-953, 2008), transposon vectors (Woltjen K, Michael I P, Mohseni P, et al: Nature 458, 766-770, 2009; Kaji K, Norrby K, Pac a A, et al: Nature 458, 771-775, 2009: Yusa K, Rad R, Takeda J, et al: Nat Methods 6,363-369, 2009), or episomal vectors (Yu J, Hu K, Smuga-Otto K, Tian S, et al: Science 324, 797-801, 2009).

Cells transformed to iPS cells, that is, cells that have undergone initialization (reprogramming) can be selected using, as an indicator, the expression of pluripotent stem cell markers (undifferentiated markers) such as Fbxo15, Nanog, Oct3/4, Fgf-4, Esg-1, and Cript, or the like. The selected cells are collected as the iPS cell.

iPS cells can be provided from FUJIFILM Cellular Dynamics, Inc.; National University Corporation, Kyoto University; or Independent Administrative Institution, Institute of Physical and Chemical Research, BioResource Research Center.

The pluripotent stem cell according to the embodiment of the present invention can be produced by introducing a DNA encoding a reporter molecule into the pluripotent stem cell described above, adjacent to the endogenous tau gene. In a case where the DNA encoding a reporter molecule is introduced adjacent to the endogenous tau gene, the tau protein can be expressed as a fusion protein fused with a reporter molecule in the pluripotent stem cells according to the embodiment of the present invention.

The endogenous tau gene may be any one of a wild-type tau gene or a mutant-type tau gene; however, it is preferably a wild-type tau gene.

Tau (microtubule-associated protein tau, also called MAPT) is a protein encoded by an MAPT gene (official full name: microtubule-associated protein Tau, official symbol: MAPT, NG_007398.1) located on chromosome 17 (17q21.1) in humans, and six isoforms produced by selective splicing have been identified.

The number (0 to 2) of characteristic amino acid sequences (N) of 29 amino acids on the N-terminal side and the number (3 or 4) of repetitive sequences (R) involved in microtubule binding on the C-terminal side are different between the isoforms, and thus depending on the number of these sequences, each of the isoforms is classified into a 0N3R type (352 amino acids, NP_058525.1, NM_016841.4), a 1N3R type (381 amino acids, NP_001190180.1, NM_001203251.1), a 2N3R type (410 amino acids, NP_001190181.1, NM_001203252.1), a 0N4R type (383 amino acids, NP 058518.1, NM_016834.4), a 1N4R type (412 amino acids, NP_001116539.1, NM_001123067.3), and a 2N4R type (441 amino acids, NP_005901.2, NM_005910.5) (regarding each of the human isoforms, the number of amino acid residues, the ID of the reference amino acid sequence, and the ID of the reference nucleotide sequence of the transcript are shown in parentheses as an example).

The tau gene in the present invention may be a tau gene of a mammal (for example, rodents such as a mouse and a rat, or primates such as a marmoset) other than the human. There are the following six types of tau protein isoforms that can be generated by alternative splicing in the brain of the mammalian living body: the 0N3R type, the 1N3R type, the 2N3R type, the 0N4R type, 1N4R type, and the 2N4R type, which are described above.

Only 3R type tau is expressed in the human brain in the fetal period. However, all of the above 6 types are expressed in the human adult brain, and the expression ratio (=4 repeat tau/3 repeat tau) of the 4R type (4 repeat tau) to the 3R type (3 repeat tau) in normal humans is about 1.

Regarding mice, only 3R-type tau is expressed up to the newborn mouse stage; however, only 4R-type tau is expressed after the weaning period.

Regarding rats and marmosets as well, only 3R-type tau is expressed up to the newborn rat or marmoset stage; however, only 4R-type tau is expressed after the weaning period.

In the present invention, unless otherwise specified, the tau may be any one of the 3 repeat tau or the 4 repeat tau.

The endogenous tau gene may be a mutant-type tau gene. Examples of the mutant-type tau gene include a mutant-type tau gene identified from the FTDP-17 family. The known tau mutation includes (i) a mutation by which the amino acid sequence of tau protein is changed and (ii) a mutation by which the expression ratio of the 4 repeat tau to the 3 repeat tau is increased or decreased.

The mutation of the type (i) described above is generally represented by an amino acid mutation that occurs in the tau protein based on the amino acid sequence of the type 2N4R (441 amino acids, NP_005901.2, NM_005910.5) which is the longest isoform. For example, the description of “P301S” means that it is a gene mutation that generates a tau protein in which the proline residue (P) at the position 301 of the amino acid sequence of NP_005910.5 is substituted with a serine residue (S), and “K280Δ” means a genetic mutation (that is, encoding a mutant tau protein) that generates a tau protein in which the lysine residue at the position 280 of the above sequence is deleted. Since the mutation sites of the type (i) identified from the FTDP-17 family are concentrated in exons 9 to 13, a tau gene having a mutation in exons 9 to 13 may be used. Examples of the mutation include one or more mutations selected from A152T, K257T, T260V, G272V, N297K, K280Δ, L284L, N296N, P301L, P301S, S305N, S305S, V337M, E342V, G389R, and R406W.

As the mutation of the type (ii) described above, a large number of mutations in intron 10 have been identified from the FTDP-17 family, and such a mutant-type tau gene may be used. Examples of the base sequence of the intron 10 of the wild-type tau gene include a base sequence consisting of bases from the 120,983th to 124,833th bases of NG_007398.1. Among the above, a tau gene having one or more mutations in a stem and loop, which is formed during splicing, and in the vicinity of the stem-loop, that is, in the 1st to 20th nucleotides of the intron 10, is preferable, and examples of such a tau gene include an MAPT gene in which the 3rd, 11th, 12th, 13th, 14th, 16th, or 19th base is substituted.

The nerve cell according to the embodiment of the present invention may be a nerve cell obtained by inducing differentiation of a pluripotent stem cell having a mutant-type tau gene. Examples of the pluripotent stem cell having a mutant-type tau gene include a pluripotent stem cell prepared from a somatic cell of an animal that endogenously has a mutant-type tau gene.

Examples of the reporter molecule include proteins that can be detected by visualization, such as an enzyme that catalyzes luminescence such as luciferase, a chromogenic protein, a luminescent protein, and a fluorescent protein. As the reporter molecule, a detection system based on intragenic complementation, such as bimolecular fluorescence complementation (BiFC) or NanoLuc (registered trade name) (chemiluminescence), may be used. In the BiFC, a fluorescent protein is divided into two parts and the parts are respectively fused with two proteins to be analyzed. In a case where the two fusion proteins come close to each other in the cell, the three-dimensional structure of the fluorescent protein is reconstructed to generate fluorescence. In the NanoLuc (registered trade name) 2 molecular technology (NanoBiT: NanoLuc (registered trade name) Binary Technology), each of Large BiT (LgBiT; 18 kDa) and Small BiT (SmBiT; an 11 amino acid peptide) subunits is expressed as a fusion protein with a target protein, and protein-protein interaction occurs, whereby subunit complementation is prompted to form a luminescent enzyme that generates bright light. The reporter molecule is preferably a fluorescent protein. The kind of fluorescent protein is not particularly limited, and any fluorescent protein can be used. Specific examples of the fluorescent protein include the following proteins. It is noted that BFP means blue fluorescent protein, CFP means cyan fluorescent protein, GFP means green fluorescent protein. YFP means yellow fluorescent protein, and RFP means red fluorescent protein.

TABLE 1

| Fluorescence | |
|---|---|
| color | Protein name |
| Blue | Sirius, EBFP, TagBFP |
| Cyan | ECFP, mTurquoise, TagCFP, AmCyan, |
| | AmCyan1, mTFP1, MidoriishiCyan, CFP |
| Green | TurboGFP, AcGFP, TagGFP, TagGFP2, |
| | Azami-Green, ZsGreen, EmGFP, EGFP, |
| | GFP2, HyPer |
| Yellow | TagYFP, EYFP, Venus, YFP, PhiYFP, |
| | PhiYFP-m, TurboYFP, ZsYellow, mBanana |
| Orange | KusabiraOrange, mOrange, mOrange2, TurboRFP |
| Red | TurboRFP, DsRed-Express, DsRed-Express2, |
| | DsRed2, TagRFP, DsRed-Monomer, AsRed2, |
| | mStrawberry, mCherry, tdTomato |
| FarRed | TurboFP602, TurboFP635, mRFP1, JRed, KillerRed, |
| | mCherry, HcRed, KeimaRed, mRasberry, mPlum, |
| | mKate2, HCRed1 |
| Cyan and green | PS-CFP |
| Green and red | Dendra2, Kaede, EosFP, KikumeGR |

As the method of introducing a DNA encoding a reporter molecule, adjacent to the endogenous tau gene, it is possible to use a genome editing technique such as zinc finger nuclease (ZFN). TALEN, or CRISPR/Cas9; however, CRISPR/Cas9 can be preferably used. CRISPR-Cas9 is a technique for causing double-strand breaks in a specific DNA strand having a base sequence complementary to a guide RNA (gRNA) by introducing a guide RNA that recognizes a targeted site on the genome and the Cas9 nuclease into a cell. The gRNA is preferably a single guide RNA (sgRNA). The Cas9 and the sgRNA need to be introduced into the nucleus in the cell in order to access the endogenous tau gene in the nucleus, which is the target of genome editing. As the vector for the introduction, a plasmid vector or a viral vector can be used. The Cas9 and the sgRNA can be introduced into a cell by an electroporation method, a lipofection method, or the like.

The DNA encoding a reporter molecule may be located upstream of the endogenous tau gene or may be located downstream of the endogenous tau gene; however, it is preferably located upstream of the endogenous tau gene. That is, the reporter molecule may be fused to the N-terminal side of the tau protein or may be fused to the C-terminal side of the tau protein; however, it is preferably fused to the N-terminal side of the tau protein. In a case where the reporter molecule is fused to the N-terminal side of the tau protein, the expression of the tau and the reporter molecule becomes strong, and thus the detection of tau by fluorescence observation or the like becomes easier. The DNA encoding a reporter molecule and the endogenous tau gene may have or may not have a linker as long as they are linked and expressed as one protein. The linker is preferably 100 bases or less, more preferably 70 bases or less, and most preferably 3 bases or less, since there is a possibility that separation occurs within the linker. The amino acid after the linker is translated is preferably 50 amino acids or less, more preferably 30 amino acids or less, and most preferably 1 amino acid or less.

The targeting vector needs to have homology with sequences in the tau gene, upstream and downstream of the vicinity (the N-terminal or the C-terminal) of the tau gene cleavage site that is determined by the gRNA sequence. The upstream homology sequence is called a left homology arm, and the downstream homology sequence is called a right homology arm. The left homology arm and the right homology arm are around 1,600 bp in total. Each of them is preferably about 800 bp. The homology arms do not have to be exactly the same as the tau gene sequence, and it is preferable that a silent mutation is introduced in the gRNA recognition sequence. The targeting vector has a DNA encoding a reporter molecule to be inserted into the tau gene, in the insides of the left homology arm and the right homology arm. It is preferable that in addition to the DNA encoding a reporter molecule, a DNA encoding a drug resistance gene or fluorescent protein is included. It is more preferable that a drug resistance gene or a gene encoding a fluorescent protein is arranged to have a PiggyBac sequence at both ends thereof. The drug resistance gene includes a puromycin resistance gene, a G418 resistance gene, a hygromycin resistance gene, a blasticidin S resistance gene, and the like. A hygromycin resistance gene is preferable. The fluorescent protein includes those listed in Table 1; however, an mRFP resistance gene is preferable. Further, it is preferable that the targeting vector has a ganciclovir susceptibility gene.

The gRNA is a sequence that determines the cleavage site of the tau gene and contains a target sequence that recognizes the N-terminal or C-terminal sequence of the tau gene. The target sequence is preferably set to about 20 bases downstream of the PAM sequence (NGG). The gRNA is preferably a sgRNA.

The present invention provides a kit including a targeting vector that includes homology arms upstream and downstream of a tau gene insertion site and includes a DNA encoding a reporter molecule; and a gRNA that determines a cleavage site of the tau gene.

In one example, the homology arm upstream of the tau gene insertion site is a sequence having 90% or more (preferably 95% or more and more preferably 97% or more) identity with a sequence set forth in SEQ ID NO: 1, and the homology arm downstream of the tau gene insertion site is a sequence having 90% or more (preferably 95% or more and more preferably 97% or more) identity with a sequence set forth in SEQ ID NO: 2. In another example, the homology arm upstream of the tau gene insertion site is a sequence having 90% or more (preferably 95% or more and more preferably 97% or more) identity with a sequence set forth in SEQ ID NO: 3, and the homology arm downstream of the tau gene insertion site is a sequence having 90% or more (preferably 95% or more and more preferably 97% or more) identity with a sequence set forth in SEQ ID NO: 4.

Preferably, the gRNA targets a sequence having 90% or more (preferably 95% or more and more preferably 97% or more) identity with a sequence set forth in SEQ ID NO: 5 or 6.

The pluripotent stem cell into which a DNA encoding a reporter molecule has been introduced can be cultured using a medium suitable for culturing pluripotent stem cells. In a case where an iPS cell is used as the pluripotent stem cell, it is possible to use, for example, StemFit (registered trade name) AK02N (Ajinomoto Co., Inc.), mTeSR (registered trade name) 1 (Stemcell Technologies), or StemFlex (registered trade name). The culture may be carried out on a plate (for example, a 6-well plate or the like) or in a flask; however, it is preferably carried out on a plate. The culture period is not particularly limited, and cells can be cultured, for example, for 1 to 10 days. It is preferably 5 to 8 days.

By collecting cells proliferated by the above culture and confirming the sequence in the vicinity of the tau gene held by the cells by DNA sequencing, it can be confirmed that a DNA encoding a reporter molecule is introduced.

Nerve Cell

The present invention relates to a nerve cell differentiated from the above pluripotent stem cell.

The nerve cell according to the embodiment of the present invention is a cell that has a DNA encoding a reporter molecule, where the DNA is introduced adjacent to an endogenous tau gene such that a tau protein is expressed as a fusion protein fused with a reporter molecule. The nerve cell according to the embodiment of the present invention may be any one an established nerve cell line or a primary nerve cell.

The nerve cell according to the embodiment of the present invention can express a fusion protein of a tau protein and a reporter molecule. Specific examples and the preferred form of the reporter molecule are as described above in the present specification. In the nerve cell according to the embodiment of the present invention, the intracellular tau can be visualized or quantified by using the expression of the reporter molecule as an indicator.

The method of inducing the differentiation of a pluripotent stem cell into a nerve cell from is not particularly limited; however, it includes a method of preparing a neural stem cell from a pluripotent stem cell by using a treatment with a low-molecular-weight compound and then inducing the neural stem cell to a nerve cell, and a method of carrying out direct induction to a nerve cell by a gene expression or the like.

Examples of the method of inducing the differentiation of a pluripotent stem cell into a nerve cell include:

(1) a method of carrying out culture in a serum-free medium to form an embryoid body (a cell mass containing neuronal precursor cells) and differentiating the embryoid body (an SFEB method: Watanabe K., et al, Nat. Neurosci., 8: 288-296, 2005; SFEBq method: Wataya T., et al, Proc. Natl. Acad. Sci. USA., 105: 11796-11801, 2008);

(2) a method of carrying out culture and differentiation on the stroma cell (an SDIA method: Kawasaki H., et al, Neuron, 28: 31-40, 2000);

(3) a method of carrying out culture and differentiation on Matrigel to which a drug has been added (Chambers S. M., et al, Nat. Biotechnol., 27: 275-280, 2009);

(4) a method carrying out culture and differentiation in a medium containing a low-molecular-weight compound as a substitute for a cytokine (U.S. Pat. No. 5,843,780A);

(5) a method of introducing a neural inducing factor (neurogenin 2 (Ngn2) or the like) into a pluripotent stem cell and expressing the nerve-inducing factor (WO2014/148646A; and Zhang Y., et al, Neuron, 78: 785-98, 2013) to carry out the differentiation of the pluripotent stem cell;

(6) a method of introducing miR-9/9*-124 into a pluripotent stem cell and expressing the miR-9/9*-124 to carry out the differentiation of the pluripotent stem cell; and

a combination of these methods.

Among them, (5) the method of introducing neurogenin 2 into a pluripotent stem cell and expressing the neurogenin 2 is preferable since mature nerve cells can be obtained in a short period of time and with high efficiency. Further, (6) the method of introducing miR-9/9*-124 into a pluripotent stem cell and expressing the miR-9/9*-124 to carry out the differentiation of the pluripotent stem cell is also preferable. The method of inducing the differentiation of a pluripotent stem cell into a nerve cell is preferably a method of carrying out direct induction to a nerve cell by expressing Ngn2 alone or Ngn2 and miR-9/9*-124. It is most preferably a method of carrying out induction to a nerve cell by expressing Ngn2 alone or Ngn2 and miR-9/9*-124 with a TET-on promoter.

The neurogenin 2 protein is a transcription factor known to promote differentiation into nerve cells during development, and the amino acid sequence thereof is exemplified by NP_076924 in humans and NP_033848 in mice. The neurogenin 2 gene (official full name: neurogenin 2, official symbol: NEUROG2, also called the Ngn2 gene) is a DNA encoding the neurogenin 2 protein, and examples thereof include NM_009718 (mouse) or NM_024019 (human) registered as the reference sequence and a DNA having a nucleotide sequence of a transcript variant thereof. Further, it may be a DNA having complementarity to the extent by which the DNA can be hybridized to the reference sequence or the nucleic acid having a sequence of the transcript variant under stringent conditions.

The stringent conditions can be determined based on the melting temperature (Tm) of the nucleic acid to which a complex or a probe binds. Examples of the washing conditions after hybridization typically include conditions of about “1× saline sodium citrate buffer (SSC), 0.1% SDS, 37° C.”. It is preferable that a complementary strand maintains a state of being hybridized with a target positive strand even in a case of being washed under such conditions. Further, examples of the washing conditions include conditions under which a positive strand maintains a state of being hybridized with a complementary strand even in a case of being washed under washing conditions of about “0.5×SSC, 0.1% SDS, 42° C.” as the more severe hybridization conditions and washing conditions of about “0.1×SSC, 0.1% SDS, 65° C.” as the still more severe hybridization conditions. Specific examples of such a complementary strand include a strand consisting of a base sequence having a completely complementary relationship with a base sequence of a target positive strand, and a strand consisting of a base sequence having at least 90%, preferably 95% or more, more preferably 97% or more, still more preferably 98% or more, and particularly preferably 99% or more identity with the above complementary strand.

The expression of neurogenin 2 in the pluripotent stem cell can be carried out by introducing a nucleic acid (DNA or RNA) encoding neurogenin 2 or a neurogenin 2 (protein) into the pluripotent stem cell.

The expression of miR-9/9*-124 in the pluripotent stem cell can be carried out by introducing a nucleic acid (DNA or RNA) encoding miR-9/9*-124 into the pluripotent stem cell.

In the present invention, in a case of introducing a nucleic acid encoding neurogenin 2 and a nucleic acid encoding miR-9/9*-124 into a cell, it is possible to introduce, for example, a vector such as a virus, a plasmid, or an artificial chromosome into a pluripotent stem cell by using a method such as lipofection, a method using a liposome, microinjection, or the like. Examples of the virus vector include a retrovirus vector, a lentivirus vector, an adenovirus vector, an adeno-associated virus vector, and a Sendai virus vector. In addition, the artificial chromosome vector includes, for example, a human artificial chromosome (HAC), a yeast artificial chromosome (YAC), a bacterial artificial chromosome (BAC, PAC), and the like. As the plasmid, a plasmid for mammals can be used. The vector can contain regulatory sequences for expressing the neurogenin 2 protein or the miR-9/9*-124 (a promoter, an enhancer, a ribosome binding sequence, a terminator, a polyadenylation site, and the like) and, as desired, it may further contain a drug resistance gene (for example, a kanamycin resistance gene, an ampicillin resistance gene, a puromycin resistance gene, or the like), a selection marker sequence such as a thymidine kinase gene or a diphtheria toxin gene, and a reporter gene sequence such as β-glucuronidase (GUS), FLAG, or the like. In particular, it is preferable that the nucleotide sequence encoding the amino acid sequence of the protein is functionally conjugated to an inducible promoter sequence so that the expression of the neurogenin 2 protein or miR-9/9*-124 can be rapidly induced at the desired time.

Examples of the inducible promoter include drug-responsive promoters, and suitable examples thereof include a tetracycline-responsive promoter (a CMV minimal promoter having a tetracycline-responsive sequence (TRE) in which seven tetO sequences are consecutively included). For example, a Tet-On/Off Advanced expression induction system is exemplified; however, a Tet-On system is preferable since it is desirable that the gene of interest can be expressed in the presence of tetracycline. That is, it is a system in which a reverse tetR (rtetR) and a fusion protein (rtTA) fused with VP16AD are simultaneously expressed. The Tet-On system can be available from Clontech and used. In addition, it is also possible to suitably use a cumate-responsive promoter (Q-mate system, Krackeler Scientific, Inc., National Research Council (NRC), or the like), an estrogen-responsive promoter (WO2006/129735A, and a GenoStat inducible expression system, Upstate Cell Signaling Solutions), an RSLI-responsive promoter (RheoSwitch mammal inducible expression system, New England Biolabs), or the like. Among the above, a tetracycline-responsive promoter or a cumate-responsive promoter is particularly preferable, and a tetracycline-responsive promoter is most preferable, due to the high specificity and the low toxicity of an expression-induced substance.

In a case of using a cumate-responsive promoter, it is suitable that a mode in which a CymR repressor is expressed in a pluripotent stem cell is provided together.

Further, the regulatory sequence and the regulatory factor of the above promoter (the rtTA and/or the CymR repressor, or the like) may be supplied by a vector into which the neurogenin 2 gene or miR-9/9*-124 has been introduced.

In a case of using a tetracycline-responsive promoter, it is possible to maintain the expression of neurogenin 2 or miR-9/9*-124 by culturing desired cells in a medium in a state of containing tetracycline or a derivative thereof, doxycycline (hereinafter abbreviated as DOX in the present application), for a desired period of time. In addition, in a case of using a cumate-responsive promoter, it is possible to maintain the expression of neurogenin 2 or miR-9/9*-124 by continuing to culture desired cells in a medium in a state of containing a cumate, for a desired period of time.

Even in a case of using a drug-responsive promoter, it is possible to stop the expression of neurogenin 2 or miR-9/9*-124 by removing the corresponding drug from the medium (for example, by replacing the medium with a drug-free medium).

In the present invention, in a case where a nucleic acid encoding neurogenin 2, or miR-9/9*-124 is introduced in the form of RNA, it may be introduced into pluripotent stem cells by a method such as electroporation, lipofection, or microinjection. In order to maintain the expression of neurogenin 2 or miR-9/9*-124 in cells, the introduction may be carried out a plurality of times, for example, twice, three times, four times, or five times.

In the present invention, in a case where neurogenin 2 is introduced in the form of a protein, it may be introduced into pluripotent stem cells, for example, by a method such as lipofection, a method of using fusion with a cell membrane-permeable peptide (for example, an HIV-derived TAT or a polyarginine), microinjection, or the like. In order to maintain the expression of neurogenin 2 in cells, the introduction may be carried out a plurality of times, for example, twice, three times, four times, or five times.

In the present invention, the period in which neurogenin 2 or miR-9/9*-124 is expressed in pluripotent stem cells for nerve cell induction is not particularly limited; however, in a case of human pluripotent stem cells, it is 2 days or more, preferably 3 days or more, and still more preferably 4 days or more.

After the expression induction in the method of carrying out differentiation by introducing neurogenin 2 or miR-9/9*-124, the pluripotent stem cells are preferably cultured in a medium (which is referred to as a medium for nerve differentiation) suitable for inducing differentiation into nerve cells. As such a medium, only the basal medium or a basal medium to which a neurotrophic factor is added can be used. The neurotrophic factor in the present invention is a ligand for a membrane receptor that plays an important role in the survival and the maintenance of function of nerve cells, and examples thereof include nerve growth factor (NGF), brain-derived neurotropic factor (BDNF), neurotrophin 3 (NT-3), neurotrophin 4/5 (NT-4/5), neurotrophin 6 (NT-6), basic FGF, acidic FGF, FGF-5, epidermal growth factor (EGF), hepatocyte growth factor (HGF), insulin, insulin-like growth factor 1 (IGF1), insulin-like growth factor 2 (IGF-2), glia cell line-derived neurotropic factor (GDNF), TGF-b2, TGF-b3, interleukin 6 (IL-6), ciliary neurotropic factor (CNTF), and LIF. Among these, the preferred neurotrophic factor is GDNF, BDNF, and/or NT-3.

Examples of the basal medium include a Glasgow's Minimal Essential Medium (GMEM) medium, an IMDM medium, a Medium 199 medium, an Eagle's Minimum Essential Medium (EMEM) medium, an αMEM medium, a Dulbecco's modified Eagle's Medium (DMEM) medium, a Ham's F-12 (F-12) medium, a Dulbecco's Modified Eagle Medium: Nutrient Mixture F-12 (DMEM/F-12) medium, an RPMI 1640 medium, a Fischer's medium, a Neurobasal Medium medium (Thermo Fisher Scientific, Inc.), and a mixed medium thereof.

The basal medium may contain serum or may be serum-free. As necessary, the medium may contain one or more serum substitutes, for example, Knockout Serum Replacement (KSR) (a serum substitute for FBS during ES cell culture), an N2 supplement (Thermo Fisher Scientific, Inc.), a B27 supplement (Thermo Fisher Scientific, Inc.), a B27 Plus supplement (Thermo Fisher Scientific, Inc.), a Culture One supplement (Thermo Fisher Scientific, Inc.), albumin, transferrin, apotransferrin, fatty acid, insulin, a collagen precursor, trace elements, 2-mercaptoethanol, and 3′-thiol glycerol, and may also contain one or more substances such as a lipid, an amino acid. L-glutamine, Glutamax (Thermo Fisher Scientific, Inc.), a non-essential amino acid, a vitamin, a growth factor, a low-molecular-weight compound, an antibiotic, an antioxidant, pyruvic acid, a buffer, an inorganic salt, selenic acid, progesterone, and putrescine.

In the present invention, as the medium for nerve differentiation, it is particularly preferable to use a medium obtained by adding a B27 Plus supplement, a Culture One supplement, Glutamax, dbcAMP, L-ascorbic acid, Y27632, and N—[N-(3,5-difluorophenacetyl-L-alanyl)]-(S)-phenylglycine t-butyl ester (DAPT) to Neurobasal plus Medium.

The culture temperature at the time of inducing the differentiation into the nerve cell, the culture temperature is not particularly limited; however, it is about 30° C. to 40° C. and preferably about 37° C. The culture is carried out in an atmosphere of the CO₂-containing air, where the CO₂ concentration is preferably about 2% to 5%.

The nerve cell in the present invention preferably a cell that expresses at least one of marker genes specific to the nerve cell, consisting of β-111 tubulin, NeuN, neural cell adhesion molecule (N-CAM), and microtubule-associated protein 2 (MAP2), and has β-III tubulin-positive protrusion (hereinafter, referred to as a neurite).

The more preferred nerve cell in the present invention is a morphologically mature nerve cell, and the still more preferred one is a glutamatergic nerve cell. Here, the morphologically mature nerve cell is a nerve cell in which the cell body is hypertrophic and the neurite is sufficiently extended (as a guide, the neurite length is extended to about 5 times or more of the diameter of the cell body).

Screening Method

The present invention relates to a method of screening a substance, including using the above-described pluripotent stem cell or nerve cell according to the embodiment of the present invention. Further, the present invention relates to a substance screened by the above-described screening method.

The nerve cell according to the embodiment of the present invention can be used in the screening of drug candidate substances for central nervous system diseases and the analysis of pathophysiological mechanisms. In the present invention, the evaluation of an increase or decrease in an expression level of tau or the evaluation of intracellular distribution of tau can be carried out based on the expression of a reporter molecule. Preferably, the increase or decrease in the expression level of tau can be evaluated based on the expression intensity of the reporter molecule. In a case of using a fluorescent protein as the reporter molecule, it is possible to carry out the evaluation of the increase or decrease in the expression level of tau or the evaluation of the intracellular distribution of tau by using the expression of tau fused with the fluorescent protein based on the expression of fluorescence, and thus it is possible to simply carry out the screening of substances such as drugs.

The nerve cell according to the embodiment of the present invention can be subjected to the monitoring of the expression of the reporter molecule in real time in a state of being alive. The instrument that can be used for the monitoring includes a fluorescence microscope, a confocal microscope, a charge coupled device (CCD) camera, and the like. In addition, in a case where real-time monitoring is not required, it is possible to observe nerve cells after fixing them with formalin or the like. Further, it is possible to carry out staining with an antibody against tau or fluorescent protein after the fixation; however, an analysis without staining is desirable since the cell detachment and the variation in the staining due to the fixation and staining operations may affect the quantitative results.

The present invention provides a method of screening a prophylactic or therapeutic drug for tauopathy, including, for example:

(1) a step of bringing the nerve cell according to the embodiment of the present invention into contact with a test substance;

(2) a step of measuring the tau expression level in the above nerve cell; and

(3) a step of selecting the test substance as a prophylactic drug or therapeutic drug for tauopathy in a case where a tau expression level measured in the step (2) in a case where the nerve cell has been brought into contact with the test substance in the step (1) is a value lower than a tau expression level in a case where the nerve cell has not been brought into contact with the test substance in the step (1).

A case of a value lower than the tau expression level in a case where the nerve cell has not been brought in contact with the test substance in the step (1) is not particularly limited as long as the value is lower; however, a test substance having an action of reducing the tau expression level to preferably 80% or less and more preferably 50% or less by bringing the nerve cell into contact with the test substance is selected in a case where the tau expression level in a case where the nerve cell has not been brought into contact with the test substance is set to 100%.

The screening method according to the embodiment of the present invention is useful in screening a compound or lead compound that is capable of being a prophylactic or therapeutic drug for tauopathy.

Examples of the tauopathy include Alzheimers disease (AD), frontotemporal dementia with Parkinsonism linked to chromosome 17 (FTDP-17), frontotemporal dementia (FTD), Pick's disease, progressive supranuclear palsy (PSP), corticobasal degeneration (CBD), argyrophilic grain dementia (argyrophilic grain disease), neurofibrillary tangle type dementia, and diffuse neurofibrillary tangles with calcification (DNTC).

Examples of the test substance include a protein, a peptide, an antibody, a non-peptide compound, a synthetic compound, a synthetic low-molecular-weight compound, a natural compound, a cell extract, a plant extract, an animal tissue extract, plasma, an extract derived from a marine organism, a cell culture supernatant, and a microbial fermentation product.

In addition, the test substance can be obtained by using any one of many approaches in combinatorial library methods known in the related art, including (1) a biological library method, (2) a synthetic library method using a deconvolution, (3) a one-bead one-compound library method, and (4) an affinity chromatography selection. The biological library method using affinity chromatography selection is limited to peptide libraries; however, other approaches can be applied to low-molecular-weight compound libraries of peptides, non-peptide oligomers, or compounds (Lam (1997) Anticancer Drug Des. 12: 145-67). Examples of the method of synthesizing molecule libraries can be found in the art (DeWitt et al. (1993) Proc. Natl. Acad. Sci. USA 90: 6909-13; Erb et al. (1994) Proc. Natd. Acad. Sci. USA 91: 11422-6; Zuckermann et al. (1994) J. Med. Chem. 37: 2678-85; Cho et al. (1993) Science 261: 1303-5; Carell et al. (1994) Angew. Chem. Int. Ed. Engl. 33:2059; Carell et. al. (1994) Angew. Chem. Int. Ed. Engl. 33:2061; Gallop et al. (1994) J. Med. Chem. 37: 1233-51). The compound libraries can be prepared as solutions (see Houghten (1992) Bio/Techniques 13: 412-21) or beads (Lam (1991) Nature 354: 82-4), chips (Fodor (1993) Nature 364: 555-6), bacteria (U.S. Pat. No. 5,223,409A), spores (U.S. Pat. Nos. 5,571,698A, 5,403,484A, and 5,223,409A), plasmids (Cull et al. (1992) Proc. Natl. Acad. Sci. USA 89:1865-9), or phages (Scott and Smith (1990) Science 249: 386-90; Devlin (1990) Science 249: 404-6; Cwirla et al. (1990) Proc. Natl. Acad. Sci. USA 87: 6378-82; Felici (1991) J. Mol. Biol. 222: 301-10; US2002/0103360A).

In the present invention, bringing a test substance into contact with the nerve cell may be carried out by adding the test substance to the culture solution of the nerve cell. The contact time is not particularly limited as long as the change of the indicator, such as fluorescence, can be confirmed; however, it is, for example, 1 day or more, 2 days or more, 3 days or more, 4 days or more, 5 days or more, 6 days or more, or 7 days or more. The concentration of the test substance to be added can be appropriately adjusted depending on the kind (in terms of solubility, toxicity, or the like) of the compound.

The culture medium of the nerve cell, which is used in a case where a test substance is brought into contact with the nerve cell, is not particularly limited as long as it is a medium in which the nerve cell is capable of being cultured; however, examples thereof include the above-described medium for nerve differentiation.

The culture temperature at the time of bringing a test substance with the nerve cells is not particularly limited; however, it is about 30° C. to 40° C. and preferably about 37° C. The culture is carried out in an atmosphere of the CO₂-containing air, where the CO₂ concentration is preferably about 2% to 5%.

The present invention will be more specifically described using Examples below; however, the present invention is not limited by Examples.

### EXAMPLES

(1) Preparation of iPS Cell Having TagGFP2-Tau Gene by Gene Editing

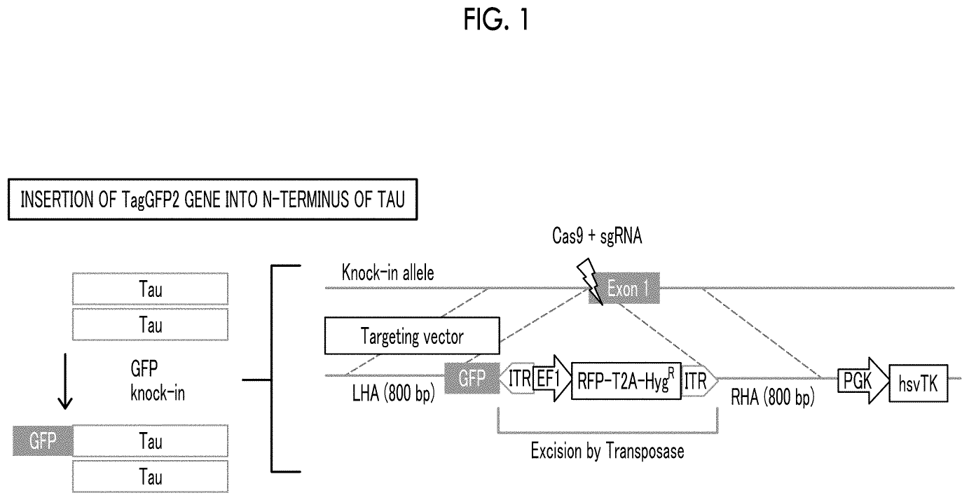

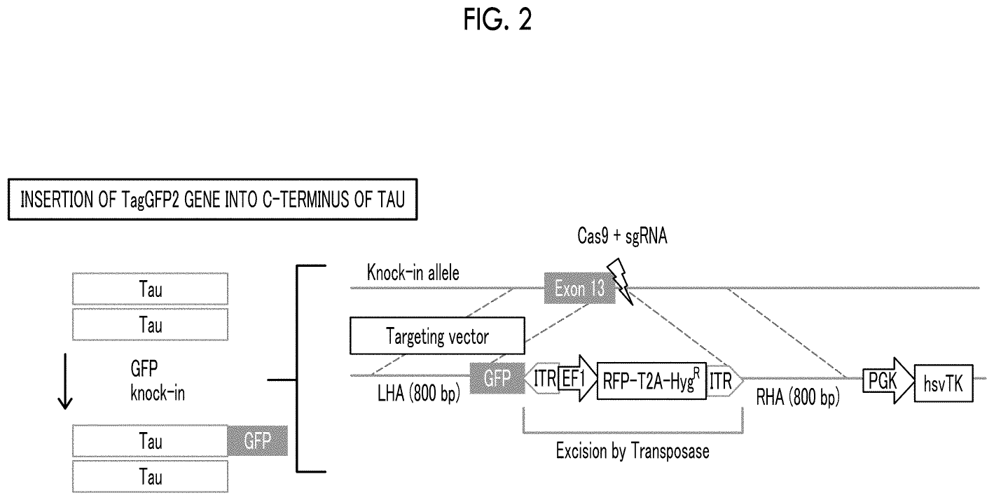

A human iPS cell 201B7 strain derived from a healthy subject (obtained from iPS Academia Japan, Inc.) was subjected to the gene editing for the tau gene. The human iPS cells were plated on a 6-well plate coated with iMatrix (Nippi, incorporated) and subjected to the feeder-free culture using StemFit (registered trade name) AK02N (Ajinomoto Co., Inc.). The cultured human iPS cells were detached with TrypLE select (Thermo Fisher Scientific, Inc.), and a cell pellet was obtained by centrifugation. The cell pellet was mixed with Neon buffer R (containing a sgRNA, a Cas9 protein, and a targeting vector) and then the introduction was carried out by electroporation using Neon (Thermo Fisher Scientific, Inc.). The targeting vector was prepared by inserting homology arms (LHA and RHA) homologous to the tau gene, a fluorescent protein gene sequence to be inserted (TagGFP2), and a gene region (EF1-RFP-T2A-Hyg) that is used for strain selection and is sandwiched by PiggyBac sequence (5′ ITR and 3′ ITR) into an HR710PA-1 vector (purchased from System Biosciences, LLC) (FIG. 1 and FIG. 2). The base sequences of the homology arms (LHA and RHA) homologous to the tau gene are shown below. In addition, the sequences of the sgRNAs (Thermo Fisher Scientific, Inc.) that recognize the genomic sequence to be cleaved are shown below.

Left homology arm (for inserting TagGFP2 into N-terminus)
(SEQ ID NO: 1)
TTTTGCTGCCAGTTGACATCTGATTGAACCATCTCTTCACTTCTCCGTGCCT
CACTTTCCTTACCAGACAGGCTCTGCTGATGCTGTCCCTCTCCTGTTCAGTCGTGCC
CTCACCGTTAAAGAGAAAGAGCAAACTGCTGGGCAGCAGCATTGATTTTTTTAATG
AAGTGGAAAGAGAGCTGGGAATAACAAGTCGGGCCCACCTCACCTGCCTCACCTG
GTGGGTTTATTTGTTTTGTTTTTTTTTTTTTGTTTTGAGACAGAGTTTCACCCTGTCA
CCCAGGCTGGAGTGCAGTGGTGTAATCTCAGCTCACTGCAACCTCCACCTGCCAGG
TTCAATTGATTCTCCTGCCTCAGCCTCCCCAGTAGCTGGGATTACAGGCACCTGCCA
CATGCCTGGCTAATTATTGTATTTTTAGTAGAGATGGGGTTTTACCATGTTGGCCAGG
CTGGTCTCGATCCCCTGACCTCAGGTGATCCACCCACCTCGGCCTCCCAAAGTGCT
GAGATCACAGGCGTGAGCCACCATGCCTGGCCGTCACCTGGTGGTGTTGAATATGA
ACTGCTGCGGTGTTGGTAAATTAAGCAAGCAGATAGATGTAAATAACGCTTGGGCA
GGAATATGGAGCACGGGATGAGGATGGGCGGCCAACTGTTAGAGAGGGTAGCAGG
GAGGCTGAGATCTGCCTGCCATGAACTGGGAGGAGAGGCTCCTCTCTCTCTTCACC
CCCACTCTGCCCCCCAACACTCCTCAGAACTTATCCTCTCCTCTTCTTTCCCCAGGT
GAACTTTGAACCAGG
Right homology arm (for inserting TagGFP2 into N-terminus)
(SEQ ID NO: 2)
ATGGCTGAGCCCCGCCAGGAGTTCGAAGTGAIGGAAGATCACGCTGGGAC
GTACGGGTTGGGGGACAGGAAAGATCAGGGGGGCTACACCATGCACCAAGACCAA
GAGGGTGACACGGACGCTGGCCTGAAAGGTTAGTGGACAGCCATGCACAGCAGGC
CCAGATCACTGCAAGCCAAGGGGTGGCGGGAACAGTTTGCATCCAGAATTGCAAA
GAAATTTTAAATACATTATTGTCTTAGACTGTCAGTAAAGTAAAGCCTCATTAATTTG
AGTGGGCCAAGATAACTCAAGCAGTGAGATAATGGCCAGACACGGTGGCTCACGC
CTGTAATCCCAGCACTTTGGAAGGCCCAGGCAGGAGGATCCCTTGAGGCCAGGAA
TTTGAGACCGGCCTGGGCAACATAGCAAGACCCCGTCTCTAAAATAATTTAAAAAT
TAGCCAGGTGTTGTGGTGCATGTCTATAGTCCTAGCTACTCAGGATGCTGAGGCAG
AAGGATCACTTGAGCCCAGGAGTTCAAGGTTGCAGTAAGCTGTGATTATAAAACTG
CACTCCAGCCTGAGCAACAGAGCAAGACCCTGTCAAAAAAAAAAGAAAAGAAAA
AAGAAAGAAAGAAATTTACCTTGAGTTACCCACATGAGTGAATGTAGGGACAGAG
ATTTTAGGGCCTTAACAATCTCTCAAATACAGGGTACTTTTTGAGGCATTAGCCACA
CCTGTTAGCTTATAAATCAGTGGTATTGATTAGCATGTAAAATATGTGACTTTAAACA
TTGCTTTTTATCTCTTACTTAGATC
Left homology arm (for inserting TagGFP2 at C-terminus)
(SEQ ID NO: 3)
ATACTAAGCAGCCTGTGTATCTATACACTCACACGTGTTTGTTTATGTGTGG
AATATCTCTGGAGGGTACACAAGAAACTTAAAATGATCACTGTCTCTGGGGAGGGT
ACCTGGGTGCCTGGGAGGCAGGTCAGGGAAGGAGTGGGCACAGGTATTACCAATT
GGAAGACAATAAAAACAACAGCTCCTGGCCAGGCGCAGTGGCTCACGCCTGTAAT
GGCAGCACTCTGAGAGGCTGAGGCGGGCAGATTGCTTGCGTCCAGGAGTTCAAGA
CCAGCCTGGGCAACATAGCAAAACCCCGTTTCTATTAAAAATACAAAAAATTAGCC
AGGTGTGGTGGCATGCACCTGTAATCCCAGCTACTCGGGAGGCTGAGGTGGGAGA
ATCACCTGAGCCTGGGAGGTCAAGGCTGCAGTGAGGTGAGATTGTGCCACCGCAC
TCTAGCCTGGGCGATAGAGCAAGACCCTGTCTCAAAAACAAACAAAAAACAGTCC
CTGGCACTCTGGGCCAGGCCTGGCAGGGCAGTTGGCAGGGCTGGTCTTTCTCTGG
CACTTCATCTCACCCTCCCTCCCTTCCTCTTCTTGCAGATTGAAACCCACAAGCTGA
CCTTCCGCGAGAACGCCAAAGCCAAGACAGACCACGGGGCGGAGATCGTGTACA
AGTCGCCAGTGGTGTCTGGGGACACGTCTCCACGGCATCTCAGCAATGTCTCCTCC
ACCGGCAGCATCGACATGGTAGACTCGCCCCAGCTCGCCACGCTAGCTGACGAGG
TGTCTGCCTCCCTGGCCAAGCAGGGTTTG
Right homology arm (for inserting TagGFP2 at C-terminus)
(SEQ ID NO: 4)
TCAGGCCCCTGGGGCGGTCAATAATTGTGGAGAGGAGAGAATGAGAGAGT
GTGGAAAAAAAAAGAATAATGACCCGGCCCCCGCCCTCTGCCCCCAGCTGCTCCT
CGCAGTTCGGTTAATTGGTTAATCACTTAACCTGCTTTTGTCACTCGGCTTTTGGCTC
GGGACTTCAAAATCAGTGATGGGAGTAAGAGCAAATTTGATCTTTCCAAATTGATG
GGTGGGCTAGTAATAAAATATTTAAAAAAAAACATTCAAAAACATGGCCACATCCA
ACATTTCCTCAGGCAATTCCTTTTGATTCTTTTTTCTTCCCCCTCCATGTAGAAGAGG
GAGAAGGAGAGGCTCTGAAAGCTGCTTCTGGGGGATTTCAAGGGACTGGGGGTGC
CAACCACCTCTGGCCCTGTTGTGGGGGTGTCACAGAGGCAGTGGCAGCAACAAAG
GATTTGAAACTTGGTGTGTTCGTGGAGCCACAGGCAGACGATGTCAACCTTGTGTG
AGTGTGACGGGGGTTGGGGTGGGGCGGGAGGCCACGGGGGAGGCCGAGGCAGGG
GCTGGGCAGAGGGGAGAGGAAGCACAAGAAGTGGGAGTGGGAGAGGAAGCCAC
GTGCTGGAGAGTAGACATCCCCCTCCTTGCCGCTGGGAGAGCCAAGGCCTATGCCA
CCTGCAGCGTCTGAGCGGCCGCCTGTCCTTGGTGGCCGGGGGTGGGGGCCTGCTG
TGGGTCAGTGTGCCACCCTCTGCAGGGCAGCCTGTGGGAGAAGGGACAGCGGGTA
AAAAGAGAAGGCAAGCTGGCAGGAGGGTG
N-TagGFP2 Tau sgRNA:
(SEQ ID NO: 5)
AGGTGAACTTTGAACCAGGA
C-TagGFP2 Tau sgRNA:
(SEQ ID NO: 6)
ACAATTATTGACCGCCCCAG

After the electroporation treatment, StemFit (registered trade name) was added and centrifuged. The cell pellet was suspended in StemFit (registered trade name) (including 10 μmol/L Y27632) and cultured in a 6-well plate coated with iMatrix. On the 3rd day of culture, the medium was replaced with StemFit (registered trade name) containing 100 μg/mL hygromycin, and then on the 5th day of culture, the medium was replaced with StemFit (registered trade name) to which 2.5 μg/mL of ganciclovir (FUJIFILM Wako Pure Chemical Corporation) had been added. The proliferated colonies were picked up under a microscope to obtain clones derived from the single cell, and then the sequence of the tau gene possessed by the cell was checked by DNA sequencing to confirm that the target sequence was inserted. Further, in order to remove the drug resistance gene sequence, an Excision only Piggybac vector (purchased from System Biosciences, LLC) was introduced by lipofection using GeneJuice (Merck KGaA), and the culture was carried out using StemFit (registered trade name). Colonies were picked up under a microscope to obtain clones derived from the single cell. Then, the sequence of the tau gene possessed by the cell was checked by DNA sequencing to confirm that the target sequence was inserted, and iPS cell clones having the TagGFP2-Tau gene were selected.

(1) Preparation of Lentivirus

A lentivirus was prepared according to the method described in Mitsuru Ishikawa, et al., Cells 2020, 9, 532. The brief description of the preparation was as follows. Three kinds of plasmids of a packaging construct (pCAG-HIVgp), VSV-G, and a Rev expression construct (pCMV-VSV-G-RSV-Rev), a self-inactivating (SIN) lentivirus vector construct (CSIV-miR-9/9*-124-mRFP1-TRE-EF-BsdT) were transfected into HEK293T cells by using polyethyleneimine (Polysciences, Inc.) to produce a lentivirus. Further, the culture supernatant was concentrated by ultracentrifugation to concentrate the lentivirus. After the concentration, the titer was measured by using lenti-Gostix PLUS (Takara Bio Inc.) and the lentivirus was used in the experiment.

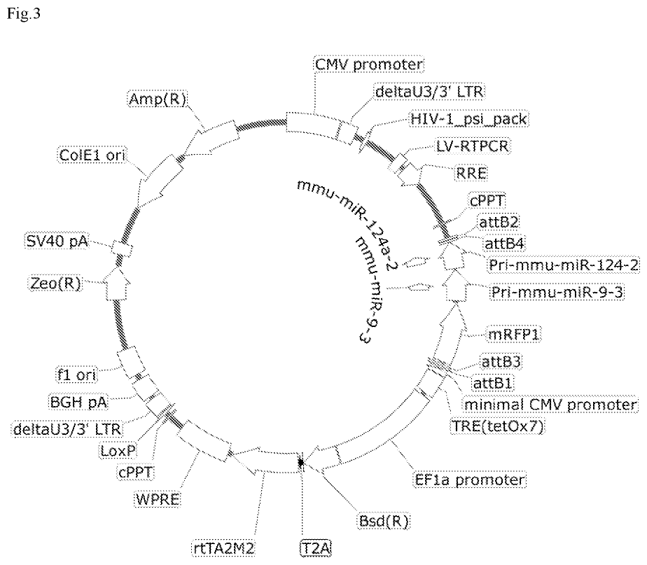

CSIV-miR-9/9*-124-mRFP1-TRE-EF-BsdT is shown in FIG. 3.

(2) Introduction of TET-on Inducible Ngn2 and miR-9/9*-124 Vectors into iPS Cell

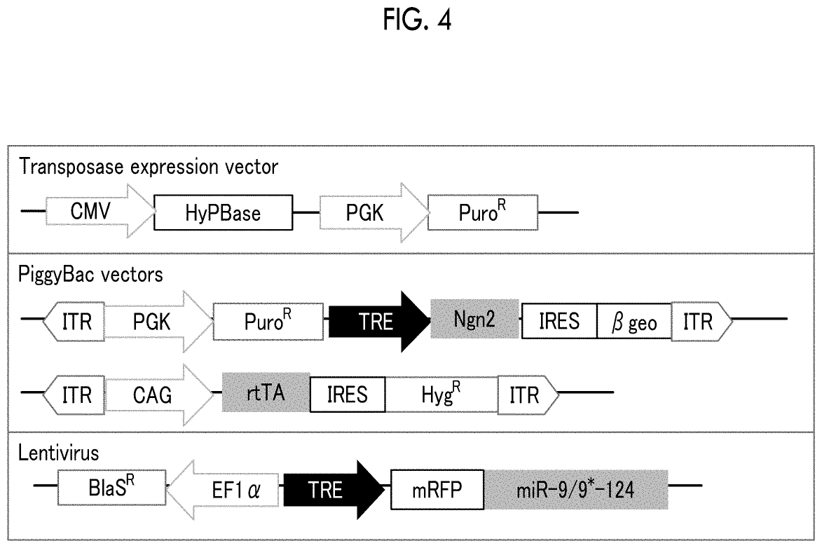

According to the method of Mitsuru Ishikawa, et al., Cells 2020, 9, 532, a transposase vector (pCMV-HyPBase-PGK-Puro) as shown in FIG. 4, a rtTA vector (PB-CAGrtTA3G-IH), and a neurogenin 2 (Ngn2) vector (PB-TET-PH-lox66FRT-NEUROG2) were prepared. These vectors were introduced into an iPS cell cultured with StemFit (registered trade name) by lipofection using GeneJuice (Merck KGaA), and further, a lentivirus (CSIV-miR-9/9*-124-mRFP1-TRE-EF-BsdT) containing the miR-9/9*-124 gene was introduced into the iPS cells. Then, iPS cell lines into which the vectors were stably introduced were obtained by drug selection using puromycin, hygromycin, and blasticidin S.

(3) Confirmation of TagGFP2-Tau Fluorescence in iPS Cell-Derived Nerve Cell

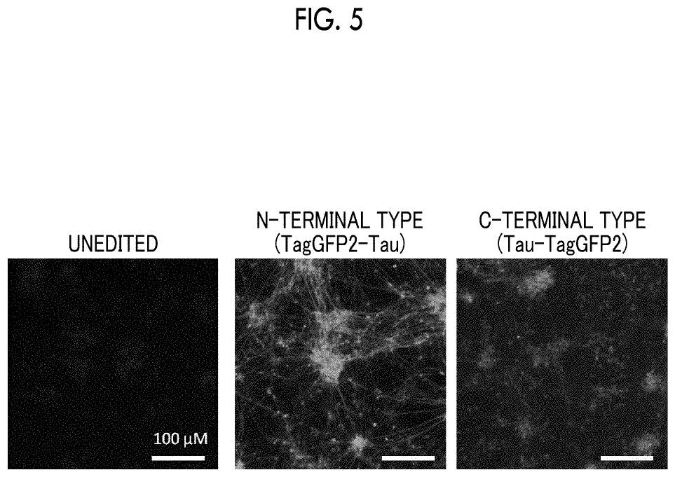

The iPS cells prepared in (2) were detached with TrypLE select and plated on a 96-well plate or 384-well plate coated with Poly-D-lysine (PDL) and iMatrix. The cells were cultured in a neural induction medium containing doxycycline (a Neurobasal plus medium (Thermo Fisher Scientific, Inc.) to which 2% of a B27 Plus supplement (Thermo Fisher Scientific, Inc.) 1% of a Cell Culture One supplement (Thermo Fisher Scientific, Inc.), 200 μmol/L of bcAMP, 200 μmol/L of L-ascorbic acid, 10 μmol/L of Y27632, 10 μmol/L of N—[N-(3,5-difluorophenacetyl-L-alanyl)]-(S)-phenylglycine t-butyl ester (DAPT), and 4 μg/ml of DOX were added), to induce differentiation into nerve cells. From the 6th day after the start of differentiation induction, the medium was replaced with a neural maintenance medium (a Neuro basal plus medium to which 2% of a B27 Plus supplement, 1% of a Culture One supplement, 200 μmol/L of dbcAMP, 200 μmol/L of L-ascorbic acid, and 10 ng/mL of brain-derived neurotropic factor (BDNF) were added), and then after the culture for 19 days, the cells were observed with a fluorescence microscope. Unedited iPS cell-derived nerve cells were observed as a negative control. The results are shown in FIG. 5. As shown in FIG. 5, fluorescence was observed in both N-terminal type (TagGFP2-Tau) nerve cells and C-terminal type (Tau-TagGFP2) nerve cells. The N-terminal type (TagGFP2-Tau) nerve cells had stronger fluorescence than the C-terminal type (Tau-TagGFP2) nerve cells.

(4) Analysis of Fluorescent Tau Quantification and Neurite Length in Bright Field

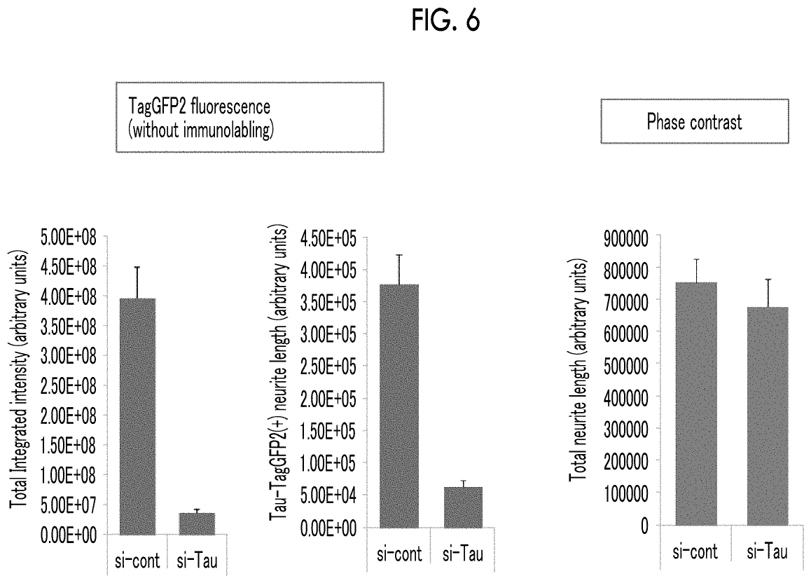

Nerve cells on the 5th day of neural induction were detached with TrypLE Select. The cells were plated to a cell number of 1×10⁴ cells/well on a 96-well plate coated with PDL and iMatrix. For culture, a neural replating medium (a medium obtained by adding 2% of a B27 Plus supplement, 1% of a Culture One supplement, 200 μmol/L of dbcAMP, 200 μmol/L of L-ascorbic acid, and 2 μg/mL of DOX to a Neurobasal plus:DMEM/F12 HAM=1:1 medium) was used to carry out culture. In addition, nerve cells were treated with 1 to 2 μmol/L of Tau Accell siRNA (Dharmacon, Inc., #D-001910-03) and cultured for about 2 weeks. Live cells were subjected to fluorescence imaging (excitation: 485 nm/emission: 535 nm) and bright field imaging of TagGFP2-Tau using In Cell Analyzer 6000 (GE Healthcare). The tau expression in the nerve cell was quantified by using the fluorescence intensity in the neurite or the positive neurite length as an indicator of the tau expression level. In addition, the nerve cell toxicity was quantified with the neurite length from bright field imaging. The results are shown in FIG. 6. As shown in FIG. 6, it was possible to detect that the amount of tau in the nerve cell was decreased since the TagGFP2 fluorescence intensity and the TagGFP2-positive protrusion length were decreased. Further, it was also confirmed at the same time that the phase difference protrusion length did not change clearly by the Tau siRNA treatment.

SEQUENCE LISTING

<110> FUJIFILM Corporation
<120> Pluripotent stem cell, nerve cell, and application thereof
<130> 20F0128001
<160> 6
<170> PatentIn version 3.5
<210> 1
<211> 800
<212> DNA
<213> Artificial Sequence
<220>
<223> homology arm
<400> 1

| | |
|---|---|
| ttttgctgcc agttgacatc tgattgaacc atctcttcac ttctccgtgc ctcactttcc | 60 |
| ttaccagaca ggctctgctg atgctgtccc tctcctgttc agtcgtgccc tcaccgttaa | 120 |
| agagaaagag caaactgctg ggcagcagca ttgatttttt taatgaagtg gaaagagagc | 180 |
| tgggaataac aagtcgggcc cacctcacct gcctcacctg gtgggtttat ttgttttgtt | 240 |
| tttttttttt tgttttgaga cagagtttca ccctgtcacc caggctggag tgcagtggtg | 300 |
| taatctcagc tcactgcaac ctccacctgc caggttcaat tgattctcct gcctcagcct | 360 |
| ccccagtagc tgggattaca ggcacctgcc acatgcctgg ctaattattg tatttttagt | 420 |
| agagatgggg ttttaccatg ttggccaggc tggtctcgat cccctgacct caggtgatcc | 480 |
| acccacctcg gcctcccaaa gtgctgagat cacaggcgtg agccaccatg cctggccgtc | 540 |
| acctggtggt gttgaatatg aactgctgcg gtgttggtaa attaagcaag cagatagatg | 600 |
| taaataacgc ttgggcagga atatggagca cgggatgagg atgggcggcc aactgttaga | 660 |
| gagggtagca gggaggctga gatctgcctg ccatgaactg ggaggagagg ctcctctctc | 720 |
| tcttcacccc cactctgccc cccaacactc ctcagaactt atcctctcct cttctttccc | 780 |
| caggtgaact ttgaaccagg | 800 |

<210> 2
<211> 800
<212> DNA
<213> Artificial Sequence
<220>
<223> homology arm
<400> 2

| | |
|---|---|
| atggctgagc cccgccagga gttcgaagtg atggaagatc acgctgggac gtacgggttg | 60 |
| ggggacagga aagatcaggg gggctacacc atgcaccaag accaagaggg tgacacggac | 120 |
| gctggcctga aaggttagtg gacagccatg cacagcaggc ccagatcact gcaagccaag | 180 |
| gggtggcggg aacagtttgc atccagaatt gcaaagaaat tttaaataca ttattgtctt | 240 |
| agactgtcag taaagtaaag cctcattaat ttgagtgggc caagataact caagcagtga | 300 |
| gataatggcc agacacggtg gctcacgcct gtaatcccag cactttggaa ggcccaggca | $60 |
| ggaggatccc ttgaggccag gaatttgaga ccggcctggg caacatagca agaccccgtc | 420 |
| tctaaaataa tttaaaaatt agccaggtgt tgtggtgcat gtctatagtc ctagctactc | 480 |
| aggatgctga ggcagaagga tcacttgagc ccaggagttc aaggttgcag taagctgtga | 540 |
| ttataaaact gcactccagc ctgagcaaca gagcaagacc ctgtcaaaaa aaaaagaaaa | 600 |
| gaaaaaagaa agaaagaaat ttaccttgag ttacccacat gagtgaatgt agggacagag | 660 |
| attttagggc cttaacaatc tctcaaatac agggtacttt ttgaggcatt agccacacct | 720 |
| gttagcttat aaatcagtgg tattgattag catgtaaaat atgtgacttt aaacattgct | 780 |
| ttttatctct tacttagatc | 800 |

<210> 3
<211> 800
<212> DNA
<213> Artificial Sequence
<220>
<223> homology arm
<400> 3

| | |
|---|---|
| atactaagca gcctgtgtat ctatacactc acacgtgttt gtttatgtgt ggaatatctc | 60 |
| tggagggtac acaagaaact taaaatgatc actgtctctg gggagggtac ctgggtgcct | 120 |
| gggaggcagg tcagggaagg agtgggcaca ggtattacca attggaagac aataaaaaca | 180 |
| acagctcctg gccaggcgca gtggctcacg cctgtaatgg cagcactctg agaggctgag | 240 |
| gcgggcagat tgcttgcgtc caggagttca agaccagcct gggcaacata gcaaaacccc | $00 |
| gtttctatta aaaatacaaa aaattagcca ggtgtggtgg catgcacctg taatcccagc | 360 |
| tactcgggag gctgaggtgg gagaatcacc tgagcctggg aggtcaaggc tgcagtgagg | 420 |
| tgagattgtg ccaccgcact ctagcctggg cgatagagca agaccctgtc tcaaaaacaa | 480 |
| acaaaaaaca gtccctggca ctctgggcca ggcctggcag ggcagttggc agggctggtc | 540 |
| tttctctggc acttcatctc accctccctc ccttcctctt cttgcagatt gaaacccaca | 600 |
| agctgacctt ccgcgagaac gccaaagcca agacagacca cggggcggag atcgtgtaca | 660 |
| agtcgccagt ggtgtctggg gacacgtctc cacggcatct cagcaatgtc tcctccaccg | 720 |
| gcagcatcga catggtagac tcgccccagc tcgccacgct agctgacgag gtgtctgcct | 780 |
| ccctggccaa gcagggtttg | 800 |

<210> 4
<211> 800
<212> DNA
<213> Artificial Sequence
<220>
<223> homology arm
<400> 4

| | |
|---|---|
| tcaggcccct ggggcggtca ataattgtgg agaggagaga atgagagagt gtggaaaaaa | 60 |
| aaagaataat gacccggccc ccgccctctg cccccagctg ctcctcgcag ttcggttaat | 120 |
| tggttaatca cttaacctgc ttttgtcact cggctttggc tcgggacttc aaaatcagtg | 180 |
| atgggagtaa gagcaaattt catctttcca aattgatggg tgggctagta ataaaatatt | 240 |
| taaaaaaaaa cattcaaaaa catggccaca tccaacattt cctcaggcaa ttccttttga | 300 |
| ttcttttttc ttccccctcc atgtagaaga gggagaagga gaggctctga aagctgcttc | 360 |
| tgggggattt caagggactg ggggtgccaa ccacctctgg ccctgttgtg ggggtgtcac | 420 |
| agaggcagtg gcagcaacaa aggatttgaa acttggtgtg ttcgtggagc cacaggcaga | 480 |
| cgatgtcaac cttgtgtgag tgtgacgggg gttggggtgg ggcgggaggc cacgggggag | 540 |
| gccgaggcag gggctgggca gaggggagag gaagcacaag aagtgggagt gggagaggaa | 600 |
| gccacgtgct ggagagtaga catccccctc cttgccgctg ggagagccaa ggcctatgcc | 660 |
| acctgcagcg tctgagcggc cgcctgtcct tggtggccgg gggtgggggc ctgctgtggg | 720 |
| tcagtgtgcc accctctgca gggcagcctg tgggagaagg gacagcgggt aaaaagagaa | 780 |
| ggcaagctgg caggagggtg | 800 |

<210> 5
<211> 20
<212> DNA
<213> Artificial Sequence
<220>
<223> target sequence of sgRNA
<400> 5

| | |
|---|---|
| aggtgaactt tgaaccagga | 20 |

<210> 6
<211> 20
<212> DNA
<213> Artificial Sequence
<220>
<223> target sequence of sgRNA
<400> 6

| | |
|---|---|
| acaattattg accgccccag | 20 |

What is claimed is:

1. A pluripotent stem cell comprising a DNA encoding a reporter molecule, the DNA being introduced adjacent to an endogenous tau gene such that a tau protein is expressed as a fusion protein fused with a reporter molecule.

2. The pluripotent stem cell according to claim 1,
wherein the pluripotent stem cell is a human pluripotent stem cell.

3. The pluripotent stem cell according to claim 1,
wherein the pluripotent stem cell is an induced pluripotent stem cell.

4. The pluripotent stem cell according to claim 1,
wherein the reporter molecule is a fluorescent protein.

5. The pluripotent stem cell according to claim 1,
wherein the DNA encoding the reporter molecule is located upstream of the endogenous tau gene.

6. A nerve cell differentiated from the pluripotent stem cell according to claim 1.

7. The nerve cell according to claim 6,
wherein a fusion protein of a tau protein and a reporter molecule is expressed.

8. A nerve cell comprising a DNA encoding a reporter molecule, the DNA being introduced adjacent to an endogenous tau gene such that a tau protein is expressed as a fusion protein fused with a reporter molecule.

9. The nerve cell according to claim 8,
wherein the nerve cell is an established nerve cell line or a primary nerve cell.

10. The nerve cell according to claim 8,
wherein the reporter molecule is a fluorescent protein.

11. The nerve cell according to claim 8,
wherein the DNA encoding the reporter molecule is located upstream of the endogenous tau gene.

12. A method of screening a substance, comprising using the nerve cell according to claim 6.

13. The method according to claim 12,
wherein an evaluation of an increase or decrease in an expression level of tau or an evaluation of intracellular distribution of tau is carried out based on an expression of a reporter molecule.

14. The method according to claim 12,
wherein an increase or decrease in an expression level of tau is evaluated based on an expression intensity of a reporter molecule.

15. A substance screened by the method according to claim 12.

16. A kit comprising:
a targeting vector that includes homology arms upstream and downstream of a tau gene insertion site and includes a DNA encoding a reporter molecule; and
a gRNA that determines a cleavage site of the tau gene.

17. The kit according to claim 16,
wherein the homology arm upstream of the tau gene insertion site is a sequence having 90% or more identity with a sequence set forth in SEQ ID NO: 1, and the homology arm downstream of the tau gene insertion site is a sequence having 90% or more identity with a sequence set forth in SEQ ID NO: 2, or
the homology arm upstream of the tau gene insertion site is a sequence having 90% or more identity with a sequence set forth in SEQ ID NO: 3, and the homology arm downstream of the tau gene insertion site is a sequence having 90% or more identity with a sequence set forth in SEQ ID NO: 4.

18. The kit according to claim 17,
wherein the gRNA targets a sequence having 90% or more identity with a sequence set forth in SEQ ID NO: 5 or 6.
